(19) United States
(12) Patent Application Publication
(10) Pub. No.: US 2020/0315641 A1
(43) Pub. Date: Oct. 8, 2020

(54) SHAFT FOR IN VIVO RECOVERY MECHANISM

(71) Applicant: ASAHI INTECC CO., LTD., Seto-shi (JP)

(72) Inventors: Hiroyuki NISHIHARA, Seto-shi (JP); Atsuhiro HANAOKA, Seto-shi (JP)

(73) Assignee: ASAHI INTECC CO., LTD., Seto-shi (JP)

(21) Appl. No.: 16/906,103

(22) Filed: Jun. 19, 2020

Related U.S. Application Data
(63) Continuation of application No. PCT/JP2018/009840, filed on Mar. 14, 2018.

(51) Int. Cl. A61B 17/22; A61B 17/3207

(52) U.S. Cl. CPC A61B 17/22; A61B 2017/00398; A61B 17/3207

(57) ABSTRACT

A shaft assembly for an in vivo recovery mechanism is provided with a shaft, and a side wire sparsely wound onto the outer circumference of the shaft and having protruding and recessed parts on the outer circumferential surface thereof. Therefore, when rotating the shaft assembly as part of an in vivo recovery mechanism, a substance in the body is caught between the sparsely wound side wire on the shaft and held by the protruding and recessed parts formed on the side wire, whereby performance for recovering and transporting the substance in the body can be improved.

### CROSS REFERENCE TO RELATED APPLICATION

This is a Continuation of PCT/JP2018/009840 filed Mar. 14, 2018. The disclosure of the prior application is hereby incorporated by reference herein in its entirety.

### BACKGROUND

The disclosed embodiments relate to a shaft for use in an in vivo recovery mechanism that removes a substance from a body lumen.

Conventionally, various devices have been developed that remove a substance from a body lumen of a patient. For example, in Japanese Unexamined Patent Application Publication No. 2013-138877, a device for removing a substance from a body lumen is described which includes a catheter having a proximal end, a distal end, and a catheter lumen that extends therethrough, a cutter assembly which is rotatably joined to the distal end of the catheter, and a rotatable torque shaft having a first end, which extends through the catheter lumen and is joined to the cutter assembly, and a second end which is configured so as to be joined to a rotation mechanism, wherein an outer coil is provided on an outer surface of the torque shaft, and the outer coil is spirally formed on the outer surface of the torque shaft such that a substance inside a body lumen is transported in the proximal direction when the torque shaft is rotated (see FIG. 20, etc.).

The device described in Japanese Unexamined Patent Application Publication No. 2013-138877 (hereinafter referred to as “in vivo recovery mechanism”) discharges a substance inside a body lumen (hereinafter referred to as “substance in the body”) to the outside of a patient's body by means of the outer coil spirally formed on the torque shaft, and the performance for recovering and transporting the substance in the body depends on the outer coil.

Furthermore, in procedures and surgeries, it is vital to quickly perform the work of recovering and transporting the substance in the body, and enhancements that enable the recovery and transport performance to be further improved are desired.

The disclosed embodiments have been devised to address the above problems associated with the conventional technique, and an object of the disclosed embodiments is to provide a shaft for an in vivo recovery mechanism that improves the recovery and transport performance of an in vivo recovery mechanism to enable in vivo recovery work in procedures and surgeries to be quickly and reliably performed.

### SUMMARY

In order to address the above problems, a shaft assembly for an in vivo recovery mechanism according to the disclosed embodiments includes a shaft, and a side wire sparsely wound onto an outer circumference of the shaft and having protruding and recessed parts on the outer circumferential surface thereof. When rotating the shaft assembly for an in vivo recovery mechanism, a substance in the body is caught between the sparsely wound side wire on the shaft and held by the protruding and recessed parts formed on the side wire, and the performance for recovering and transporting the substance in the body can be improved.

The side wire may include a first strand formed by twisting a plurality of first wires, and the protruding and recessed parts of the side wire may be formed from an outline of the first strand. The protruding and recessed parts of the side wire are easily formed, and when rotating the shaft for an in vivo recovery mechanism, a substance in the body is caught between the sparsely wound side wire on the shaft and held by the protruding and recessed parts formed from the outline of the first strand, and the performance for recovering and transporting the substance in the body can be improved.

A twisting direction of the first strand may be the same direction as a winding direction of the side wire onto the shaft, and the protruding and recessed parts may extend in a direction that is inclined with respect to the longitudinal axis of the shaft, and when rotating the shaft assembly for an in vivo recovery mechanism, in addition to the effects described above, the performance for recovering and transporting the substance in the body can be further improved.

The protruding and recessed parts may be formed on an outer circumferential surface of the wires constituting the first strand, and therefore, in addition to the effects described above, the performance for recovering and transporting the substance in the body can be further improved.

The protruding and recessed parts may extend along a direction substantially perpendicular to the longitudinal axis of the shaft, and therefore, when rotating the shaft assembly for an in vivo recovery mechanism, in addition to the effects described above, the performance for recovering and transporting the substance in the body can be further improved.

The shaft assembly may include a second strand formed by twisting a plurality of second wires, and therefore, in addition to the effects described above, the shaft assembly for an in vivo recovery mechanism is made softer, and further, when rotating the shaft assembly for an in vivo recovery mechanism, the substance in the body is caught between the sparsely wound side wire on the shaft and held by the protruding and recessed parts formed from the second strand and the protruding and recessed parts formed on the side wire, and the performance for recovering and transporting the substance in the body can be further improved.

The shaft assembly may include a hollow strand formed by twisting a plurality of third wires, and therefore, in addition to the effects described above, the shaft assembly for an in vivo recovery mechanism is made even softer, and further, when rotating the shaft assembly for an in vivo recovery mechanism, the substance in the body is caught between the sparsely wound side wire on the shaft and held by the protruding and recessed parts formed from the hollow strand of the shaft and the protruding and recessed parts formed on the side wire, and the performance for recovering and transporting the substance in the body can be further improved. Furthermore, by inserting a guide wire or the like into a void inside the hollow strand and causing the hollow strand to be positioned along the guide wire, the shaft for an in vivo recovery mechanism is capable of reaching the periphery of a body lumen such as a blood vessel.

### BRIEF DESCRIPTION OF THE DRAWINGS

FIG. 1 is an external view of an in vivo recovery mechanism using a shaft assembly for an in vivo recovery mechanism according to the disclosed embodiments.

FIG. 2 is an explanatory diagram of the inside of section A in FIG. 1.

FIG. 3 is an enlarged view of the shaft assembly for an in vivo recovery mechanism shown in FIG. 1.

FIG. 4 is an enlarged view of a shaft assembly for an in vivo recovery mechanism according to the disclosed embodiments.

FIG. 5 is an enlarged view of a shaft assembly for an in vivo recovery mechanism according to the disclosed embodiments.

FIG. 6 is an enlarged view of a shaft assembly for an in vivo recovery mechanism according to the disclosed embodiments.

FIG. 7 is an enlarged view of a shaft assembly for an in vivo recovery mechanism according to the disclosed embodiments.

FIG. 8 is an enlarged view of a shaft assembly for an in vivo recovery mechanism according to the disclosed embodiments.

FIG. 9 is an enlarged view of a shaft assembly for an in vivo recovery mechanism according to the disclosed embodiments.

### DETAILED DESCRIPTION OF EMBODIMENTS

Hereinafter, embodiments of the present invention will be described with reference to the drawings.

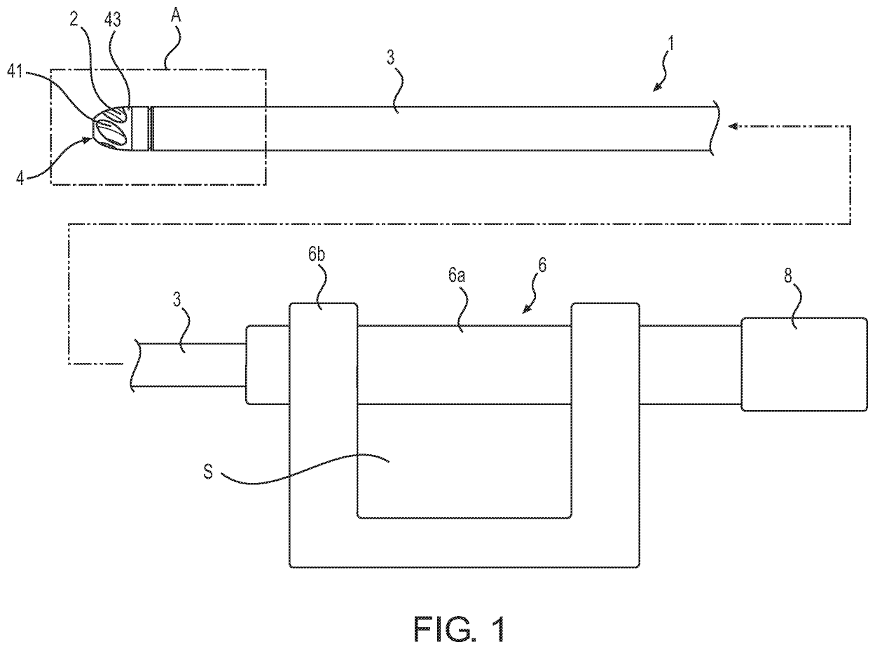

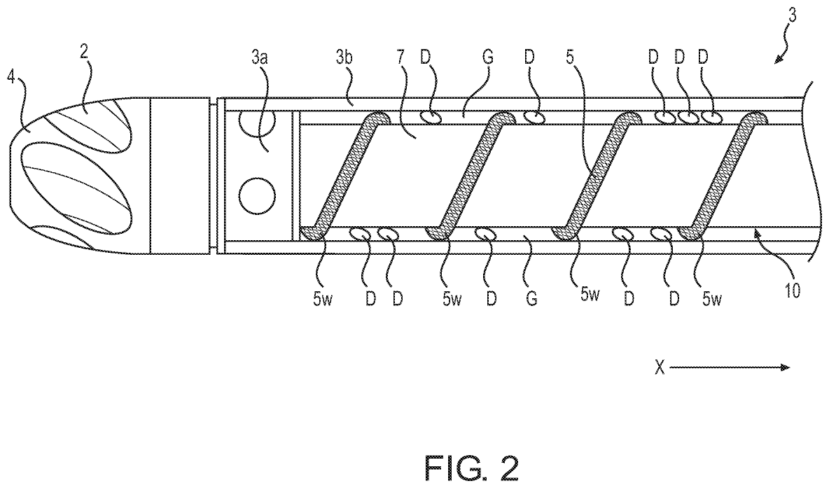

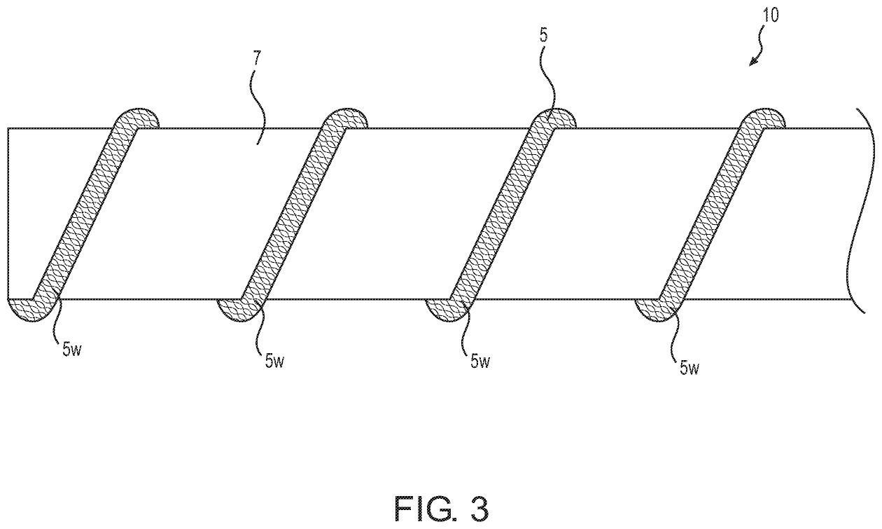

FIG. 1 is an external view of an in vivo recovery mechanism using a shaft assembly for an in vivo recovery mechanism according to the disclosed embodiments, FIG. 2 is an explanatory diagram of the inside of section A in FIG. 1, and FIG. 3 is an enlarged view of the shaft assembly shown in FIG. 1.

As shown in FIG. 1, an in vivo recovery mechanism 1 of the disclosed embodiments for removing a substance from a body lumen includes a catheter 3 having a long hollow tubular body, a cutter assembly 4 connected to the distal end of the catheter 3, a grip part 6 connected to the proximal end of the catheter 3, and a motor 8 connected to the proximal end of the grip part 6.

The cutter assembly 4 includes a plurality of openings 41, a casing 43 connected to the distal end of the catheter 3, and a cutter 2 disposed inside the casing 43. The cutter 2 is connected to the distal end of the shaft assembly 10 for an in vivo recovery mechanism described below (see FIG. 2), and is capable of rotating with the rotation of the motor 8.

Therefore, the cutter assembly 4 cuts a substance in the body D (see FIG. 2), such as plaque, that has entered from the openings 41 of the casing 43 by means of the rotating cutter 2, and then takes the substance into the interior of the cutter assembly 4.

The grip part 6 is connected to the proximal end of the catheter 3, and includes a grip part main body 6a, and a grip part side body 6b, which is letter U-shaped and is connected to the grip part main body 6a. Furthermore, a gap S, which allows for gripping of the grip part 6 by the person performing the procedure, is formed in the grip part 6 by the grip part main body 6a and the grip part side body 6b.

Moreover, the casing of the motor 8 is connected to the proximal end of the grip part 6, and the rotation shaft of the motor 8 is connected to the proximal end of the shaft 10 for an in vivo recovery mechanism described below (see FIG. 2).

In FIG. 1, the cutter assembly 4 is linearly connected to the catheter 3, and although the mechanism is not shown, in reality the cutter assembly 4 is capable of being bent by an operation of the grip part 6 in all 360 degrees with respect to the longitudinal axis of the catheter 3.

The catheter 3 includes a catheter main body 3b, a bearing 3a connected to the interior of the distal end of the catheter main body 3b, and a bearing (not shown) connected to the interior of the proximal end of the catheter main body 3b. Furthermore, the catheter 3 is connected at its distal end to the cutter assembly 4, and is connected at its proximal end to the grip part 6. The interior of the catheter 3 is provided with the rotatable shaft assembly 10 for an in vivo recovery mechanism.

As shown in FIG. 2, the shaft assembly 10 for an in vivo recovery mechanism is constituted by a shaft 7 and a side wire 5 wound onto the outer circumference of the shaft 7, and is capable of rotating inside the catheter 3 with the rotation of the motor 8. The side wire 5 is sparsely wound, meaning that there is a large winding gap between each coil turn (winding).

The shaft 7 is formed from a single long metallic wire, and is connected to the proximal end of the cutter 2 through the bearing 3a, which is connected to the interior of the distal end of the catheter main body 3b. The material of the shaft 7 is not particularly limited as long as it is a biocompatible material such as stainless steel, a Ni—Ti alloy, or a cobalt alloy.

The shaft 7 may be formed from a long solid metal wire, or may be formed from a long hollow metal wire. However, the shaft 10 for an in vivo recovery mechanism can be made softer by forming the shaft 7 with a long hollow metallic wire, and by inserting a guide wire or the like into a void inside the hollow shaft and causing the hollow shaft to be positioned along the guide wire, the shaft 10 for an in vivo recovery mechanism is capable of reaching the periphery of a body lumen such as a blood vessel.

Fine protruding and recessed parts 5w are formed on the outer circumferential surface of the side wire 5, and the area in which the protruding and recessed parts 5w are formed is indicated by a hatching pattern in FIG. 2 and FIG. 3. That is to say, the protruding and recessed parts 5w are formed on the entire outer circumferential surface of the side wire 5 of the shaft assembly 10 shown in FIGS. 1-3.

Although the protruding and recessed parts 5w as described above are formed on the entire outer circumferential surface of the side wire 5, they may be formed on only a part of the outer circumferential surface of the side wire 5. However, when the protruding and recessed parts 5w are formed on the entire outer circumferential surface of the side wire 5, it is possible to improve the retention performance of the substance in the body and improve the recovery and transport performance to a greater extent.

The material of the side wire 5 is not particularly limited as long as it is a biocompatible material such as stainless steel, tungsten, or a Ni—Ti alloy.

Moreover, because a gap G is formed between the catheter 3 and the shaft assembly 10 for an in vivo recovery mechanism, the substance in the body D, such as plaque, which is cut by the cutter 2 and taken into the interior of the cutter assembly 4, is retained inside the gap G, and is transported in the X direction by the rotation of the shaft assembly 10 for an in vivo recovery mechanism.

The shaft assembly 10 for an in vivo recovery mechanism is provided with the shaft 7, and the side wire 5 sparsely wound onto the outer circumference of the shaft 7 and having protruding and recessed parts 5w on the outer circumferential surface thereof, and therefore, when rotating the shaft assembly 10 for an in vivo recovery mechanism, the substance in the body D is caught between the sparsely wound side wire 5 on the shaft 7 and held by the protruding and recessed parts 5w formed on the side wire 5, and the performance for recovering and transporting the substance in the body can be improved.

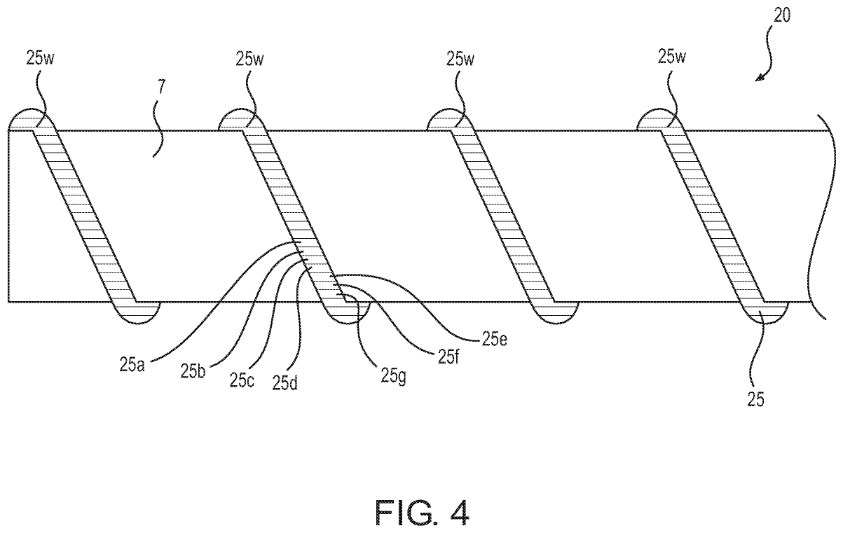

FIG. 4 is an enlarged view of a shaft assembly for an in vivo recovery mechanism according to the disclosed embodiments.

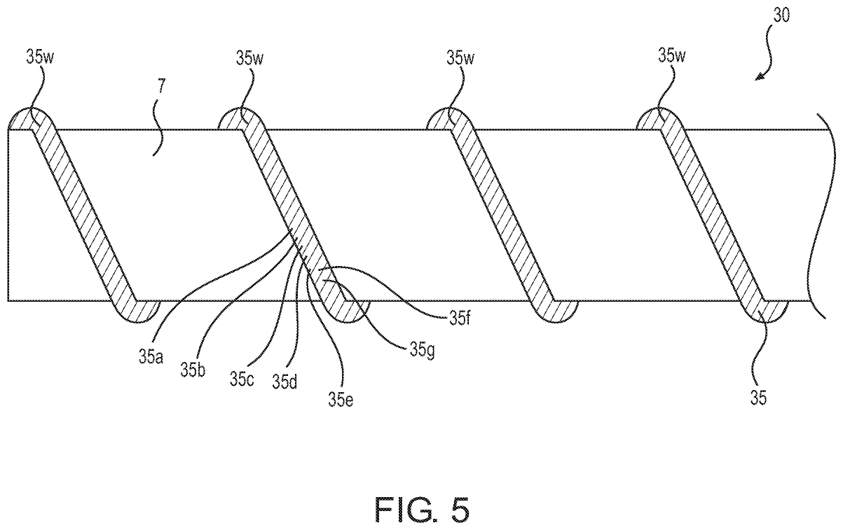

The in vivo recovery mechanism used with the shaft assembly shown in FIG. 4 (and FIGS. 5-9) is identical to that shown in FIG. 1, and the interior of the distal end of the in vivo recovery mechanism is identical to that shown in FIG. 2, and therefore the description thereof will be omitted. And throughout this disclosure, parts that are the same as described previously will be referred to by the same reference numerals, and the description will be omitted.

The shaft assembly 20 for an in vivo recovery mechanism shown in FIG. 4 is connected at its distal end to the cutter 2 through the bearing 3a connected to the interior of the distal end of the catheter main body 3b, and is connected at its proximal end to the motor 8 through a bearing (not shown) connected to the interior of the proximal end of the catheter main body 3b, and is capable of rotating with the rotation of the motor 8.

As shown in FIG. 4, the shaft assembly 20 for an in vivo recovery mechanism includes the shaft 7 and a strand 25 (corresponding to a “first strand” of the disclosed embodiments) serving as the side wire sparsely wound onto the outer circumference of the shaft 7. The strand 25 is formed by twisting seven wires (corresponding to “first wires” of the disclosed embodiments) 25a, 25b, 25c, 25d, 25e, 25f, and 25g.

Furthermore, the twisting direction of the strand 25 is a counterclockwise direction (hereinafter, referred to as “S-twisting” direction) toward the left direction in the drawing, and the winding direction of the strand 25 onto the shaft 7 is a clockwise winding (hereinafter, referred to as “Z-winding” direction) toward the left direction of the drawing, such that the twisting direction of the strand 25 and the winding direction of the strand 25 onto the shaft 7 are opposite directions.

Protruding and recessed parts 25w are formed from the outline of the strand 25 (the contour of the twisted first wires), and the protruding and recessed parts 25w are formed around the entire outer circumferential surface of the strand 25.

Furthermore, the material of the first wires 25a, 25b, 25c, 25d, 25e, 25f, and 25g constituting the strand 25 is not particularly limited as it is a biocompatible material such as stainless steel, tungsten, or a Ni—Ti alloy.

The shaft assembly 20 for an in vivo recovery mechanism is provided with the shaft 7, and the strand 25 sparsely wound onto the outer circumference of the shaft 7 and having protruding and recessed parts 25w on the outer circumferential surface thereof, and therefore, the protruding and recessed parts 25w of the strand 25 can be easily formed, and when rotating the shaft assembly 20 for an in vivo recovery mechanism, the substance in the body D is caught between the sparsely wound strand 25 on the shaft 7 and held by the protruding and recessed parts 25w formed from the outline of the strand 25, and the performance for recovering and transporting the substance in the body can be improved.

Note that, as shown in FIG. 4, the strand 25 serving as the side wire is a strand consisting of seven wires, however the number of wires is not limited to seven, and the strand may include any number of two or more wires. However, from the viewpoint of the effect of the protruding and recessed parts 25w, it is better to form the strand using the largest possible number of wires.

FIG. 5 is an enlarged view of a shaft assembly for an in vivo recovery mechanism according to the disclosed embodiments.

The shaft assembly 30 for an in vivo recovery mechanism shown in FIG. 5 is connected at its distal end to the cutter 2 through the bearing 3a connected to the interior of the distal end of the catheter main body 3b, and is connected at its proximal end to the motor 8 through a bearing (not shown) connected to the interior of the proximal end of the catheter main body 3b, and is capable of rotating with the rotation of the motor 8.

As shown in FIG. 5, the shaft assembly 30 for an in vivo recovery mechanism includes the shaft 7 and a strand 35 (corresponding to a “first strand” of the disclosed embodiments) serving as the side wire sparsely wound onto the outer circumference of the shaft 7. The strand 35 is formed by twisting seven wires (corresponding to “first wires” of the disclosed embodiments) 35a, 35b, 35c, 35d, 35e, 35f, and 35g.

Furthermore, the twisting direction of the strand 35 is a clockwise direction (hereinafter, referred to as “Z-twisting” direction) toward the left direction of the drawing, and the winding direction of the strand 35 onto the shaft 7 is a Z-winding direction, such that the twisting direction of the strand 35 and the winding direction of the strand 35 onto the shaft 7 are the same direction.

Moreover, protruding and recessed parts 35w are formed from the outline of the strand 35, and the protruding and recessed parts 35w are formed around the entire outer circumferential surface of the strand 35. In addition, the protruding and recessed parts 35w (which respectively correspond to the first wires and the grooves between the first wires) each extend in a direction that is inclined with respect to the longitudinal axis of the shaft 7, when viewed in a cross section extending in a plane along the longitudinal axis of the shaft 7.

The material of the first wires 35a, 35b, 35c, 35d, 35e, 35f, and 35g constituting the strand 35 is not particularly limited as long as it is a biocompatible material such as stainless steel, tungsten, or a Ni—Ti alloy.

Because the twisting direction of the strand 35 serving as the side wire and the winding direction of the strand 35 onto the shaft 7 are the same direction, and the protruding and recessed parts 35w extend in a direction that is inclined with respect to the longitudinal axis of the shaft 7, then when rotating the shaft assembly 30 for an in vivo recovery mechanism, the performance for recovering and transporting the substance in the body can be further improved.

Note that, in FIG. 5, the strand 35 serving as the side wire is a strand consisting of seven wires, however the number of wires is not limited to seven, and the strand may include any number of two or more wires. However, from the viewpoint of the effect of the protruding and recessed parts 35w, it is better to form the strand using the largest possible number of wires.

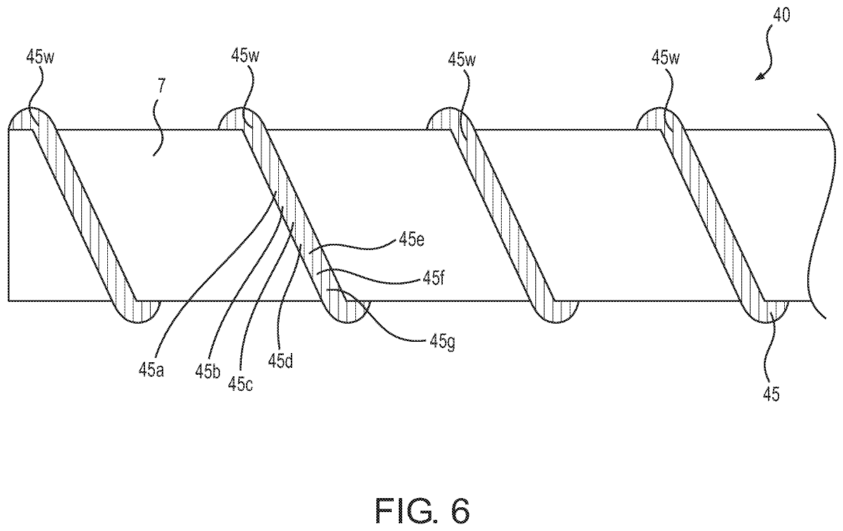

FIG. 6 is an enlarged view of a shaft assembly for an in vivo recovery mechanism according to the disclosed embodiments.

The shaft assembly 40 for an in vivo recovery mechanism shown in FIG. 6 is connected at its distal end to the cutter 2 through the bearing 3a connected to the interior of the distal end of the catheter main body 3b, and is connected at its proximal end to the motor 8 through a bearing (not shown) connected to the interior of the proximal end of the catheter main body 3b, and is capable of rotating with the rotation of the motor 8.

As shown in FIG. 6, the shaft assembly 40 for an in vivo recovery mechanism includes the shaft 7 and a strand 45 (corresponding to a “first strand” of the disclosed embodiments) serving as the side wire sparsely wound onto the outer circumference of the shaft 7, and the strand 45 is formed by twisting seven wires (corresponding to “first wires” of the disclosed embodiments) 45a, 45b, 45c, 45d, 45e, 45f, and 45g.

Furthermore, the twisting direction of the strand 45 is a Z-twisting direction, and the winding direction of the strand 45 onto the shaft 7 is a Z-winding direction, such that the twisting direction of the strand 45 and the winding direction of the strand 45 onto the shaft 7 are the same direction.

Moreover, protruding and recessed parts 45w are formed from the outline of the strand 45, and the protruding and recessed parts 45w are formed around the entire outer circumferential surface of the strand 45. In addition, the protruding and recessed parts 45w each extend along a direction substantially perpendicular to the longitudinal axis of the shaft 7, when viewed in a cross section extending in a plane along the longitudinal axis of the shaft 7.

The material of the first wires 45a, 45b, 45c, 45d, 45e, 45f, and 45g constituting the strand 45 is not particularly limited as long as it is a biocompatible material such as stainless steel, tungsten, or a Ni—Ti alloy.

Because the protruding and recessed parts 45w extend along a direction substantially perpendicular to the longitudinal axis of the shaft 7, when the shaft assembly 40 for an in vivo recovery mechanism is rotated, the performance for recovering and transporting the substance in the body can be further improved.

Note that, in FIG. 6, the strand 45 serving as the side wire is a strand consisting of seven wires, however the number of wires is not limited to seven, and the strand may include any number of two or more wires. However, from the viewpoint of the effect of the protruding and recessed parts 45w, it is better to form the strand using the largest possible number of wires.

Furthermore, although the protruding and recessed parts 45w can be formed from the outline of the strand 45 serving as the side wire as shown in FIG. 6, the protruding parts and recessed parts may instead be formed on the outer circumferential surface of the side wire 5 in the shaft 10 so as to extend along a direction substantially perpendicular to the longitudinal axis of the shaft 7.

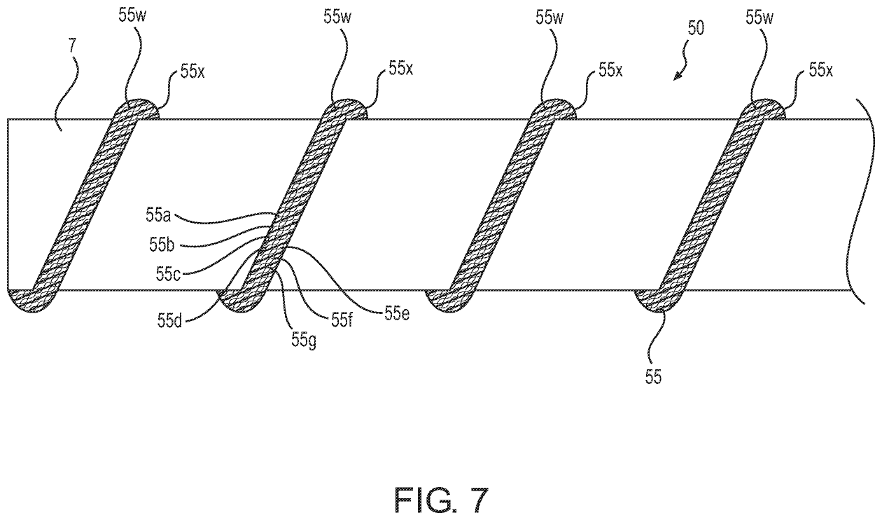

FIG. 7 is an enlarged view of a shaft assembly for an in vivo recovery mechanism according to the disclosed embodiments.

The shaft assembly 50 for an in vivo recovery mechanism shown in FIG. 7 is connected at its distal end to the cutter 2 through the bearing 3a connected to the interior of the distal end of the catheter main body 3b, and is connected at its proximal end to the motor 8 through a bearing (not shown) connected to the interior of the proximal end of the catheter main body 3b, and is capable of rotating with the rotation of the motor 8.

As shown in FIG. 7, the shaft assembly 50 for an in vivo recovery mechanism includes the shaft 7 and a strand 55 (corresponding to a “first strand” of the disclosed embodiments) serving as the side wire sparsely wound onto the outer circumference of the shaft 7, and the strand 55 is formed by twisting seven wires (corresponding to “first wires” of the disclosed embodiments) 55a, 55b, 55c, 55d, 55e, 55f, and 55g.

The twisting direction of the strand 55 is a Z-twisting direction, and the winding direction of the strand 55 onto the shaft 7 is a counterclockwise winding (hereinafter, referred to as “S-winding” direction) toward the left direction of the drawing, such that the twisting direction of the strand 55 and the winding direction of the strand 55 onto the shaft 7 are opposite directions.

Moreover, protruding and recessed parts 55w are formed from the outline of the strand 55, and fine protruding and recessed parts 55x are formed on the outer circumferential surface of the first wires 55a, 55b, 55c, 55d, 55e, 55f, and 55g constituting the strand 55.

The area in which the protruding and recessed parts 55x are formed is indicated by a hatching pattern in FIG. 7. That is to say, the protruding and recessed parts 55x shown in FIG. 7 are formed on the entire outer circumferential surface of the wires 55a, 55b, 55c, 55d, 55e, 55f, and 55g. However, they may instead be formed on part of the outer circumferential surface of the wires, or they may be formed on the entire outer circumferential surface of some of the wires and not formed at all on the outer circumferential surface of the other wires.

However, when the protruding and recessed parts 55x are formed on the entire outer circumferential surface of the wires 55a, 55b, 55c, 55d, 55e, 55f, and 55g, it is possible to improve the retention performance of the substance in the body and improve the recovery and transport performance to a greater extent.

Furthermore, the material of the first wires 55a, 55b, 55c, 55d, 55e, 55f, and 55g constituting the strand 55 is not particularly limited as long as it is a biocompatible material such as stainless steel, tungsten, or a Ni—Ti alloy.

Because the protruding and recessed parts 55w are formed from the outline of the strand 55, and the protruding and recessed parts 55x are formed on the outer circumferential surface of the wires 55a, 55b, 55c, 55d, 55e, 55f, and 55g constituting the strand 55, the performance for recovering and transporting the substance in the body can be further improved.

Note that, in FIG. 7, the strand 55 serving as the side wire is a strand consisting of seven wires, however the number of wires is not limited to seven, and the strand may include any number of two or more wires. However, from the viewpoint of the effect of the protruding and recessed parts 55w, it is better to form the strand using the largest possible number of wires.

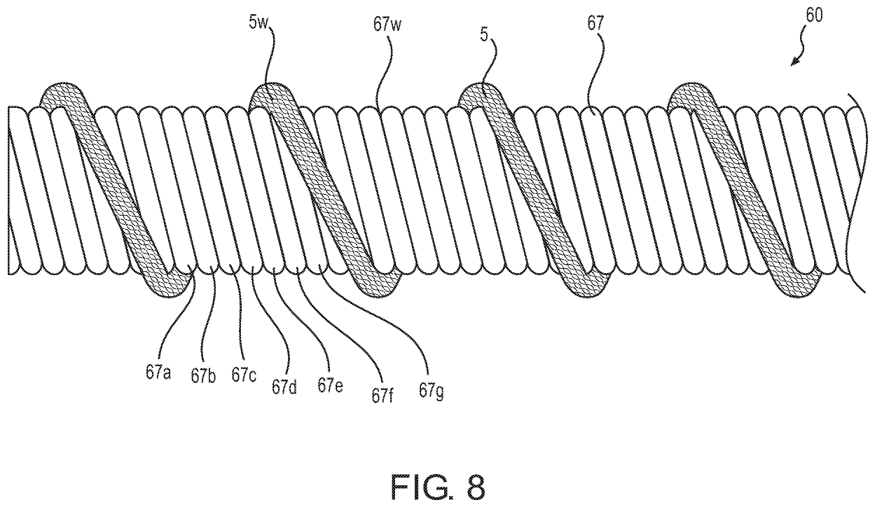

FIG. 8 is an enlarged view of a shaft assembly for an in vivo recovery mechanism according to the disclosed embodiments.

The shaft assembly 60 for an in vivo recovery mechanism shown in FIG. 8 is connected at its distal end to the cutter 2 through the bearing 3a connected to the interior of the distal end of the catheter main body 3b, and is connected at its proximal end to the motor 8 through a bearing (not shown) connected to the interior of the proximal end of the catheter main body 3b, and is capable of rotating with the rotation of the motor 8.

As shown in FIG. 8, the shaft assembly 60 for an in vivo recovery mechanism includes a strand 67, which is a shaft formed by twisting seven wires (corresponding to “second wires” of the disclosed embodiments) 67a, 67b, 67c, 67d, 67e, 67f, and 67g, and the side wire 5 sparsely wound onto the outer circumference of the strand 67.

Furthermore, the twisting direction of the strand 67 is a Z-twisting direction, and the winding direction of the side wire 5 onto the shaft 7 is a Z-winding direction, such that the twisting direction of the strand 67 and the winding direction of the side wire 5 onto the shaft 67 are the same direction.

Moreover, protruding and recessed parts 67w are formed from the outline of the strand 67.

The material of the second wires 67a, 67b, 67c, 67d, 67e, 67f, and 67g constituting the strand 67 is not particularly limited as long as it is a biocompatible material such as stainless steel, tungsten, or a Ni—Ti alloy.

Because the shaft is constituted by a strand 67 formed by twisting a plurality of wires 67a, 67b, 67c, 67d, 67e, 67f, and 67g, the shaft assembly 60 for an in vivo recovery mechanism is made softer, and further, when rotating the shaft assembly 60 for an in vivo recovery mechanism, the substance in the body D is caught between the sparsely wound side wire 5 on the strand 67 and held by the protruding and recessed parts 67w formed from the strand 67 and the protruding and recessed parts 5w formed on the side wire 5, and the performance for recovering and transporting the substance in the body can be further improved.

Note that, in FIG. 8, the strand 67 serving as the shaft is a strand consisting of seven wires, however the number of wires is not limited to seven, and of the strand may include any number of two or more wires. However, from the viewpoint of the effect of the protruding and recessed parts 67w, it is better to form the strand using the largest possible number of wires.

Furthermore, the strand 35 shown in FIG. 5, the strand 45 shown in FIG. 6, or the strand 55 shown in FIG. 7 may be used instead of the side wire 5 sparsely wound onto the outer circumference of the strand 67, and in those cases, the effects of each alternative side wire described above are provided in addition to the effects of the strand 67.

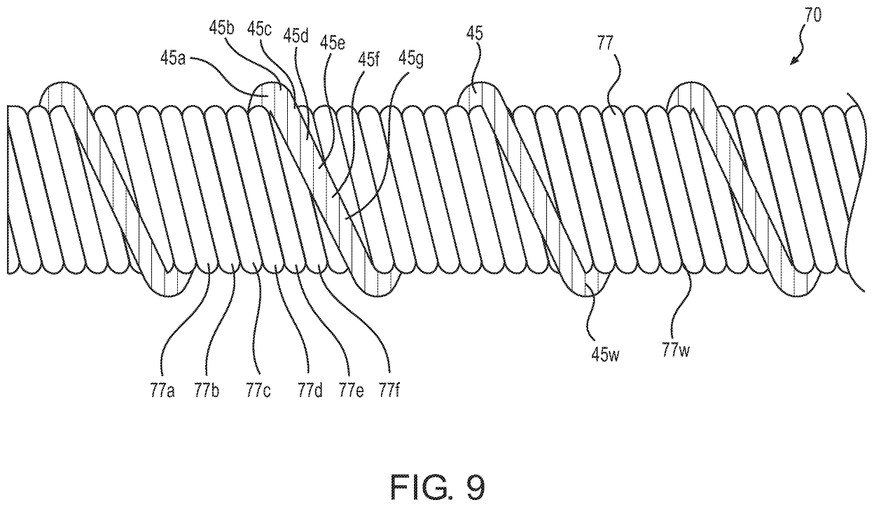

FIG. 9 is an enlarged view of a shaft assembly for an in vivo recovery mechanism according to the disclosed embodiments.

The shaft assembly 70 for an in vivo recovery mechanism shown in FIG. 9 is connected at its distal end to the cutter 2 through the bearing 3a connected to the interior of the distal end of the catheter main body 3b, and is connected at its proximal end to the motor 8 through a bearing (not shown) connected to the interior of the proximal end of the catheter main body 3b, and is capable of rotating with the rotation of the motor 8.

As shown in FIG. 9, the shaft assembly 70 for an in vivo recovery mechanism includes a hollow strand 77, which is a hollow shaft formed by twisting six wires (corresponding to “third wires” of the disclosed embodiments) 77a, 77b, 77c, 77d, 77e, and 77f, and the strand 45 sparsely wound onto the outer circumference of the hollow strand 77.

Furthermore, the twisting direction of the hollow strand 77 is a Z-twisting direction, and the winding direction of the strand 45 onto the hollow strand 77 is a Z-winding direction, such that the twisting direction of the hollow strand 77 and the winding direction of the strand 45 onto the hollow strand 77 are the same direction.

Moreover, protruding and recessed parts 77w are formed from the outline of the hollow strand 77.

In addition, the material of the third wires 77a, 77b, 77c, 77d, 77e, and 77f, constituting the hollow strand 77 is not particularly limited as long as it is a biocompatible material such as stainless steel, tungsten, or a Ni—Ti alloy.

Because the shaft is constituted by the hollow strand 77 formed by twisting the plurality of third wires 77a, 77b, 77c, 77d, 77e, and 77f, the shaft assembly 70 for an in vivo recovery mechanism is made softer, and further, when rotating the shaft assembly 70 for an in vivo recovery mechanism, the substance in the body D is caught between the sparsely wound strand 45 on the hollow strand 77 and held by the protruding and recessed parts 77w formed from the hollow strand 77 and the protruding and recessed parts 45w formed on the side wire, and the performance for recovering and transporting the substance in the body can be further improved.

Furthermore, by inserting a guide wire or the like into a void inside the hollow strand 77 and causing the hollow strand 77 to be positioned along the guide wire, the shaft assembly 70 for an in vivo recovery mechanism is capable of reaching the periphery of a body lumen such as a blood vessel.

Note that, in FIG. 9, the hollow strand 77 serving as the shaft is a strand consisting of six wires, however the number of wires is not limited to six, and of the shaft may include any number of two or more wires. However, from the viewpoint of the effect of the protruding and recessed parts 77w, it is better to form the strand using the largest possible number of wires.

Furthermore, the side wire 5 shown in FIGS. 2, 3, and 8, the strand 25 shown in FIG. 4, the strand 35 shown in FIG. 5, and the strand 55 shown in FIG. 7 may be applied instead of the strand 45 sparsely wound onto the outer circumference of the strand 77, and in those cases, the effects of each alternative side wire described above are provided in addition to the effects of the strand 77.

1. A shaft assembly for an in vivo recovery mechanism, the shaft assembly comprising:
a shaft; and
a side wire wound onto an outer circumference of the shaft so that a gap is included between each winding of the side wire, the side wire including first protruding and recessed parts on an outer circumferential surface of the side wire.

2. The shaft assembly according to claim 1, wherein:
the side wire comprises a first strand formed by twisting a plurality of first wires, and
the first protruding and recessed parts are formed from an outline of the first strand.

3. The shaft assembly according to claim 2, wherein a twisting direction of the first strand is the same as a winding direction of the side wire onto the shaft.

4. The shaft assembly according to claim 2, wherein second protruding and recessed parts are formed on an outer circumferential surface of the first wires.

5. The shaft assembly according to claim 1, wherein the first protruding and recessed parts extend along a direction substantially perpendicular to a longitudinal axis of the shaft, when viewed in a cross section extending in a plane along the longitudinal axis of the shaft.

6. The shaft assembly according to claim 2, wherein the first protruding and recessed parts extend along a direction substantially perpendicular to a longitudinal axis of the shaft, when viewed in a cross section extending in a plane along the longitudinal axis of the shaft.

7. The shaft assembly according to claim 3, wherein the first protruding and recessed parts extend along a direction substantially perpendicular to a longitudinal axis of the shaft, when viewed in a cross section extending in a plane along the longitudinal axis of the shaft.

8. The shaft assembly according to claim 4, wherein the first protruding and recessed parts extend along a direction substantially perpendicular to a longitudinal axis of the shaft, when viewed in a cross section extending in a plane along the longitudinal axis of the shaft.

9. The shaft assembly according to claim 1, wherein the shaft comprises a strand formed by twisting a plurality of wires.

10. The shaft assembly according to claim 2, wherein the shaft comprises a second strand formed by twisting a plurality of second wires.

11. The shaft assembly according to claim 3, wherein the shaft comprises a second strand formed by twisting a plurality of second wires.

12. The shaft assembly according to claim 4, wherein the shaft comprises a second strand formed by twisting a plurality of second wires.

13. The shaft assembly according to claim 5, wherein the shaft comprises a strand formed by twisting a plurality of wires.

14. The shaft assembly according to claim 1, wherein the shaft comprises a hollow strand formed by twisting a plurality of wires.

15. The shaft assembly according to claim 2, wherein
the shaft is constituted by a hollow strand formed by twisting a plurality of second wires.

16. The shaft assembly according to claim 3, wherein
the shaft is constituted by a hollow strand formed by twisting a plurality of second wires.

17. The shaft assembly according to claim 4, wherein
the shaft is constituted by a hollow strand formed by twisting a plurality of second wires.

18. The shaft assembly according to claim 5, wherein
the shaft is constituted by a hollow strand formed by twisting a plurality of second wires.

19. The shaft assembly according to claim 2, wherein a twisting direction of the first strand is opposite to a winding direction of the side wire onto the shaft.

20. The shaft assembly according to claim 1, wherein the first protruding and recessed parts extend along a direction that is inclined with respect to a longitudinal axis of the shaft, when viewed in a cross section extending in a plane along the longitudinal axis of the shaft.
